(19) United States
(12) Patent Application Publication
(10) Pub. No.: US 2013/0211110 A1
(43) Pub. Date: Aug. 15, 2013

(54) Process for manufacturing epichlorohydrin

(71) Applicants: Dominique Balthasart, Brussels (BE); Patrick Gilbeau, Braine-le-comte (BE); Philippe Krafft, Rhode Saint Genese (BE)

(72) Inventors: Dominique Balthasart, Brussels (BE); Patrick Gilbeau, Braine-le-comte (BE); Philippe Krafft, Rhode Saint Genese (BE)

(73) Assignee: SOLVAY SA, Brussels (BE)

(21) Appl. No.: 13/878,429

(22) PCT Filed: Oct. 28, 2011
(86) PCT No.: PCT/EP2011/068999

§ 371 Date: Apr. 9, 2013

(30) Foreign Application Priority Data
Oct. 29, 2010 (FR) 1058955

(51) Int. Cl. C07D 301/32

(52) U.S. Cl. CPC C07D 301/32
USPC 549/514

(57) ABSTRACT

Process for manufacturing epichlorohydrin, comprising: (a) preparing epichlorohydrin to obtain a mixture comprising epichlorohydrin and water; (b) subjecting the mixture obtained in step (a) to a liquid-liquid phase separation to separate at least one first fraction (I) comprising most of the epichlorohydrin which was contained in the mixture before separation and at least one second fraction (II) comprising most of the water which was contained in the mixture before separation; and (c) drawing off the fraction (I) and the fraction (II); wherein the volume V_{I} of the fraction (I) obtained in step (b) expressed in m³, the volume V_{II} of the fraction (II) obtained in step (b) expressed in m³, the draw-off flow rate D_{I} of the fraction (I) in step (c) expressed in m³/h, and the draw-off flow rate IN of the fraction (II) in step (c) expressed in m³/h, correspond to the following formula: (V_{II}/V_{I})<(D_{II}/D_{I}).

The present application claims benefit of French patent application n° 1058955 filed on Oct. 29, 2010, the content of which is incorporated herein by reference for all purposes.

Should the disclosure of any of the patents, patent applications, and publications which are incorporated herein by reference conflict with the description of the present application to the extent that it may render a term unclear, the present description shall take precedence.

The present invention relates to a process for manufacturing epichlorohydrin. The present invention relates more specifically to a process for manufacturing epichlorohydrin that generates a mixture comprising epichlorohydrin and water.

International application WO 2008/101866 filed in the name of SOLVAY SA discloses a process for manufacturing epichlorohydrin via reaction between dichloropropanol and a basic compound comprising a step of recovering, by settling, the epichlorohydrin formed in the mixture resulting from the reaction between the dichloropropanol and the basic compound. The conditions disclosed for the settling operation do not make it possible to avoid a certain degradation of the epichlorohydrin during this operation.

The present invention aims to overcome this problem by providing a process for manufacturing epichlorohydrin, according to which:
(a) epichlorohydrin is prepared so as to obtain a mixture comprising epichlorohydrin and water;
(b) the mixture obtained in step (a) is subjected to a liquid-liquid phase separation so as to separate at least one first fraction (I) containing most of the epichlorohydrin that was contained in the mixture obtained in step (a) before the separation and at least one second fraction (II) containing most of the water that was contained in the mixture obtained in step (a) before the separation;
(c) fraction (I) and fraction (II) are drawn off; in which the volume V_{I} of the fraction (I) obtained in step (b) expressed in m³, the volume V_{II} of the fraction (II) obtained in step (b) expressed in m³, the draw-off flow rate D_{I} of the fraction (I) in step (c) expressed in m³/h and the draw-off flow rate D_{II} of the fraction (II) in step (c) expressed in m³/h, correspond to the following formula:

(V_{II}/V_{I})<(D_{II}/D_{I})

Surprisingly, it has been observed that working under the volume and flow rate conditions of the fractions of the process according to the invention has the advantage of resulting in a better overall degree of recovery of the epichlorohydrin. Without wishing to be tied to any one theoretical explanation, it is believed that the fraction of epichlorohydrin recovered in fraction (I) and the fraction of epichlorohydrin that can be recovered in fraction (II) are higher, following limitation of the epichlorohydrin degradation reactions during the phase separation step. The epichlorohydrin that can be recovered in fraction (II) is the epichlorohydrin that can be recovered in subsequent treatment steps of fraction (II). These degradation reactions are, for example, the hydrolysis reactions of epichlorohydrin to monochloropropanediol and to glycerol. In the process according to the invention, the ratio of the volumes (V_{II}/V_{I}) is preferably less than or equal to 0.7 times the ratio of the flow rates (D_{II}/D_{I}), still preferably less than or equal to 0.5 times the ratio of the flow rates (D_{II}/D_{I}), more preferably less than or equal to 0.4 times the ratio of the flow rates (D_{II}/D_{I}), even more preferably less than or equal to 0.3 times the ratio of the flow rates (D_{II}/D_{I}), more preferably still less than or equal to 0.2 times the ratio of the flow rates (D_{II}/D_{I}) and very particularly preferably less than 0.1 times the ratio of the flow rates (D_{II}/D_{I}).

In the process according to the invention, the ratio of the volumes (V_{II}/V_{I}) is preferably greater than or equal to 0.005 times the ratio of the flow rates (D_{II}/D_{I}), more preferably greater than or equal to 0.05 times the ratio of the flow rates (D_{II}/D_{I}) and very particularly preferably greater than or equal to 0.1 times this ratio of the flow rates (D_{II}/D_{I}).

In the process according to the invention, the volume V_{I} of the fraction (I) obtained in step (b) expressed in m³, the volume V_{II} of the fraction (II) obtained in step (b) expressed in m³, the draw-off flow rate D_{I} of the fraction (I) in step (c) expressed in m³/h and the draw-off flow rate D_{II} of the fraction (II) in step (c) expressed in m³/h, correspond to the following formula:

[(V_{I}+V_{II})/(D_{I}+D_{II}]<10 h

In the process according to the invention, the sum of the volumes V_{II} and V_{I} expressed in m³ is more preferably less than or equal to 5 times the sum of the flow rates D_{II} and D_{I} expressed in m³/h, even more preferably less than or equal to 2 times the sum of the flow rates D_{II} and D_{I}, very particularly preferably less than or equal to 1 times the sum of the flow rates D_{II} and D_{I}, still very particularly preferably less than or equal to 0.8 times the sum of the flow rates D_{II} and D_{I}, yet very particularly preferably less than or equal to 0.5 times the sum of the flow rates D_{II} and D_{I} and most preferably less than or equal to 0.4 times the sum of the flow rates D_{II} and D_{I}.

In the process according to the invention, the sum of the volumes V_{II} and Vᵢ expressed in m³ is preferably greater than or equal to 0.001 times the sum of the flow rates D_{II} and D_{I} expressed in m³/h, more preferably greater than or equal to 0.01 times the sum of the flow rates D_{II} and D_{I}, more preferably greater than or equal to 0.05 times the sum of the flow rates D_{II} and D_{I} and very particularly preferably greater than or equal to 0.1 times the sum of the flow rates D_{II} and D_{I}.

In the process according to the invention, the mixture comprising epichlorohydrin and water may originate from any manufacturing process. Examples of such processes are the processes for manufacturing epichlorohydrin, the processes for manufacturing a derivative of epichlorohydrin, in particular epoxy resins, and combinations of at least two thereof. The derivatives of epichlorohydrin and the epoxy resins may be as described in application WO 2008/152044 filed in the name of SOLVAY (Societe Anonyme), of which the content, and more specifically the passage from page 13, line 22, to page 44, line 8, is incorporated herein by reference.

In the process according to the invention, the mixture comprising epichlorohydrin and water preferably originates from a process for manufacturing epichlorohydrin, from a process for manufacturing epoxy resins, or from a combination of at least two of these processes.

In the process according to the invention, the mixture comprising epichlorohydrin and water more preferably originates from a process for manufacturing epichlorohydrin, even more preferably from a process for manufacturing epichlorohydrin by dehydrochlorination of dichloropropanol, and very particularly preferably from a process for manufacturing epichlorohydrin by dehydrochlorination of dichloropropanol in which at least one portion of the dichloropropanol was obtained from glycerol and of which at least one fraction of said glycerol is natural glycerol. The dehydrochlorination of dichloropropanol is preferably an alkaline dehydrochlorination. The expression “natural glycerol” is understood to mean glycerol which has been obtained from renewable raw materials. The natural glycerol is as described in application WO 2006/100312 in the name of SOLVAY (Societe Anonyme), of which the content, and more specifically the passage from page 4, line 22, to page 5, line 24, is incorporated herein by reference.

In the process according to the invention, at least one portion of the natural glycerol was preferably obtained in the manufacture of biodiesel.

The processes for preparing dichloropropanol and epichlorohydrin can be such as disclosed in International applications WO 2005/054167, WO2006/100311, WO2006/100312, WO2006/100313, WO2006/100314, WO2006/100315, WO2006/100316, WO2006/100317, WO2006/106153, WO2007/054505, WO 2006/100318, WO2006/100319, WO2006/100320, WO 2006/106154, W02006/106155, WO 2007/144335, WO 2008/107468, WO 2008/101866, WO 2008/145729, WO 2008/110588, WO 2008/152045, WO 2008/152043, WO 2009/000773, WO 2009/043796, WO 2009/121853, WO 2008/152044, WO 2009/077528, WO 2010/066660, WO 2010/029039, WO 2010/029153, WO 2011/054769 and WO 2011/054770, filed in the name of SOLVAY, the contents of which are incorporated herein by reference.

In the process according to the invention, the mixture obtained in step (a) comprises epichlorohydrin, water and preferably at least one salt.

In the process according to the invention, the mixture obtained in step (a) preferably comprises, in addition, at least one salt.

In the process according to the invention, when the mixture comprises epichlorohydrin, water and at least one salt, this mixture more preferably originates from a process for manufacturing epichlorohydrin as described in application WO 2008/101866 in the name of SOLVAY (Societe Anonyme), of which the content, and more specifically the passage from page 2, line 4, to page 6, line 21, is incorporated herein by reference.

In the process according to the invention, when the mixture comprises epichlorohydrin, water and at least one salt, this mixture more preferably originates from a process for manufacturing epichlorohydrin, even more preferably from a process for manufacturing epichlorohydrin by dehydrochlorination of dichloropropanol, and very particularly preferably from a process for manufacturing epichlorohydrin by dehydrochlorination of dichloropropanol in which at least one portion of the dichloropropanol was obtained from glycerol and of which at least one fraction of said glycerol is natural glycerol.

In the process according to the invention, the mixture from step (a) comprises epichlorohydrin at a content generally greater than or equal to 10 g of epichlorohydrin per kg of mixture, preferably greater than or equal to 30 g/kg, more preferably greater than or equal to 50 g/kg, even more preferably greater than or equal to 70 g/kg, more preferably still greater than or equal to 100 g/kg, particularly preferably greater than or equal to 150 g/kg and more particularly preferably greater than or equal to 170 g/kg and very particularly preferably greater than or equal to 200 g/kg. This epichlorohydrin content is generally less than or equal to 800 g of epichlorohydrin per kg of mixture, preferably less than 600 g/kg, more preferably less than or equal to 400 g/kg, even more preferably less than or equal to 500 g/kg, and very particularly preferably less than or equal to 350 g/kg.

In the process according to the invention, the mixture from step (a) comprises water at a content generally greater than or equal to 20 g of water per kg of mixture, preferably greater than 50 g/kg, more preferably greater than or equal to 100 g/kg, even more preferably greater than or equal to 200 g/kg and very particularly preferably greater than or equal to 300 g/kg. This water content is generally less than or equal to 900 g of water per kg of mixture, preferably less than 800 g/kg, more preferably less than or equal to 700 g/kg, even more preferably less than or equal to 650 g/kg and very particularly preferably less than or equal to 600 g/kg.

In the process according to the invention, when the mixture from step (a) comprises at least one salt, the salt content is generally greater than or equal to 1 g of salt per kg of mixture, preferably greater than 10 g/kg, more preferably greater than or equal to 50 g/kg, even more preferably greater than or equal to 80 g/kg, very particularly preferably greater than or equal to 90 g/kg and most preferably greater than or equal to 120 g/kg. This salt content is generally less than or equal to 250 g of salt per kg of mixture, preferably less than 220 g/kg, more preferably less than or equal to 200 g/kg, even more preferably less than or equal to 180 g/kg and very particularly preferably less than or equal to 160 g/kg.

In the process according to the invention, when the mixture from step (a) comprises at least one salt, the salt may be an organic salt, an inorganic salt or a mixture of the two. An inorganic salt is a salt whose constituent anions and cations do not contain a carbon-hydrogen bond. The inorganic salt may be chosen from the group constituted of metal chlorides, metal sulphates, metal hydrogen sulphates, metal hydroxides, metal carbonates, metal hydrogen carbonates, metal phosphates, metal hydrogen phosphates, metal borates and mixtures of at least two thereof Alkali and alkaline-earth metal chlorides are preferred. Sodium and potassium chlorides are more particularly preferred and sodium chloride is very particularly preferred. In the process according to the invention, the mixture comprising epichlorohydrin and water may contain at least one compound other than the epichlorohydrin, the water and a salt. This compound may be as described for the liquid reaction medium in application WO 2008/101866 in the name of SOLVAY (Societe Anonyme), of which the content, and more specifically the passage from page 6, line 22, to page 7, line 16, is incorporated herein by reference. This other compound is, for example, a derivative of the epichlorohydrin manufacturing process, and may be found in the group constituted of dichloropropanols, glycerol, monochloropropanediols, glycerol esters, esters of monochloropropanediols, esters of dichloropropanols, partially chlorinated and/or esterified glycerol oligomers, aldehydes such as acrolein, ketones such as chloracetone, chloroethers, basic compounds, acid compounds such as hydrogen chloride, fatty acids, and mixtures of at least two thereof. The at least one compound other than the epichlorohydrin, the water and a salt, is preferably dichloropropanol.

In the process according to the invention, when the mixture from step (a) comprises dichloropropanol, the dichloropropanol content is generally greater than or equal to 1 g of dichloropropanol per kg of mixture, preferably greater than 10 g/kg and more preferably greater than or equal to 50 g/kg. This dichloropropanol content is generally less than or equal to 200 g of dichloropropanol per kg of mixture, preferably less than 150 g/kg, more preferably less than or equal to 100 g/kg and even more preferably less than or equal to 75 g/kg.

This other compound may be a basic compound, for example when the mixture containing epichlorohydrin, water and preferably at least one salt is obtained by dehydrochlorination of dichloropropanol. This basic compound may be an organic basic compound or an inorganic basic compound or a mixture of the two. Organic basic compounds are, for example, amines, such as for example imidazole and derivatives thereof, pyridine and derivatives thereof, phosphines and ammonium, phosphonium or arsonium hydroxides. Inorganic basic compounds are preferred. The expression “inorganic compounds” is understood to mean compounds which do not contain a carbon-hydrogen bond.

The inorganic basic compound may be chosen from alkali metal oxides, hydroxides, carbonates, hydrogen carbonates, phosphates, hydrogen phosphates and borates, alkaline-earth metal oxides, hydroxides, carbonates, hydrogen carbonates, phosphates, hydrogen phosphates and borates, and mixtures of at least two thereof Alkali metal oxides, alkali metal hydroxides, alkaline-earth metal oxides, alkaline-earth metal hydroxides, and mixtures of at least two thereof, are preferred. Sodium hydroxide, calcium hydroxide and mixtures thereof are preferred. Sodium hydroxide is particularly preferred.

In one particular embodiment of the process according to the invention, the pH of the mixture obtained in step (a) is controlled and maintained at a value generally greater than or equal to 4, often greater than or equal to 5 and frequently greater than or equal to 6. This pH is controlled and maintained at a value generally less than or equal to 10, often less than or equal to 9 and frequently less than or equal to 8.

In the process according to the invention, step (b) is generally carried out in a liquid-liquid phase separation zone. Often, at least one liquid-liquid phase separation zone is fed with the mixture from (a). The expression “separation zone” is understood to mean the zone between feeding the mixture and drawing off the first fraction (I), containing most of the epichlorohydrin that was contained in the mixture obtained in step (a) before the separation, and the second fraction (II) containing most of the water, and optionally salt, which were contained in the mixture obtained in step (a) before the separation. The liquid-liquid phase separation zone may consist of any type of equipment that makes it possible to carry out a liquid-liquid separation. Such equipment is, for example, described in Perry's Chemical Engineers' Handbook, Sixth Edition, McGraw Hill, 1984, Section 21-64 and 21-68.

In the process according to the invention, the mixture comprising epichlorohydrin, water and optionally at least one salt preferably feeds a single phase separation zone, and more specifically, this zone preferably consists of a gravity-type separator. The gravity separator may be of assisted or unassisted type. When the gravity separator is of assisted type, the assistance to the gravitation may be chosen from the group constituted of centrifugal force, pulsation, coalescence, plates and combinations of at least two thereof. Examples of a centrifugal force-assisted gravity separator are a centrifugal dryer, a centrifuge and a stirred column. An example of a pulsation-assisted gravity separator is a pulsed column. An example of a coalescence-assisted gravity separator is a settler/coalescer. An example of a plate-assisted gravity separator is a plate settler. In the latter case, the plates reduce the settling height. The separator is preferably chosen from the group constituted of a gravity settling tank, a settler/coalescer, a plate settler and combinations of at least two thereof The separator is more preferably chosen from the group constituted of a gravity settling tank, a settler/coalescer and combinations thereof. The separator is more preferably a gravity settling tank.

In the process according to the invention, the liquid-liquid phase separation is carried out at a temperature generally greater than or equal to 0° C., often greater than or equal to 5° C., frequently greater than or equal to 10° C., in a lot of cases greater than or equal to 20° C. and in particular greater than or equal to 40° C. This temperature is generally less than or equal to 100° C., often less than or equal to 85° C., frequently less than or equal to 75° C. and in a lot of cases less than or equal to 50° C. In the process according to the invention, the pressure in the phase separation zone is generally greater than or equal to 0.01 bar absolute, often greater than or equal to 0.1 bar absolute, frequently greater than or equal to 0.15 bar absolute, in a lot of cases greater than or equal to 0.2 bar absolute and in particular greater than or equal to 0.6 bar absolute. This pressure is generally less than or equal to 20 bar absolute, often less than or equal to 15 bar absolute, frequently less than or equal to 10 bar absolute and in a lot of cases less than or equal to 1.5 bar absolute.

In the process according to the invention, the separation of fractions (I) and (II) is preferably carried out by unassisted gravitation or by centrifugal force-assisted gravitation or by coalescence-assisted gravitation, preferably by unassisted gravitation or by coalescence-assisted gravitation, and more preferably by unassisted gravitation. The separation may be facilitated by the use of any physical or chemical means or combinations thereof. The physical means may be of static or mechanical type or may combine the two types. A static physical means is, for example, the use of a static coalescing bed. A dynamic physical means is, for example, the use of controlled stirring. The chemical means are, for example, means that reduce the interfacial tension between the fractions to be separated or that increase the difference in density between fractions to be separated or that reduce the viscosity of the phases to be separated.

In the process according to the invention, when the mixture obtained in step (a) originates partly from a process for manufacturing epichlorohydrin by dehydrochlorination of dichloropropanol, it is possible to add dichloropropanol to the mixture obtained in step (a) so as to facilitate the phase separation of step (b).

In the process according to the invention, when the mixture obtained in step (a) originates partly from a process for manufacturing epichlorohydrin by alkaline dehydrochlorination of dichloropropanol, it is possible to add dichloropropanol to the mixture obtained in step (a) so as to facilitate the phase separation of step (b).

In the process according to the invention, when the mixture obtained in step (a) originates partly from a process for manufacturing epichlorohydrin by alkaline dehydrochlorination of dichloropropanol and in which at least one portion of the dichloropropanol was obtained from glycerol and of which at least one fraction of the glycerol is natural glycerol, it is possible to add dichloropropanol to the mixture obtained in step (a) so as to facilitate the phase separation of step (b).

In the process according to the invention, the difference in density between the fractions (I) and (II) is generally greater than or equal to 0.001, often greater than or equal to 0.002, frequently greater than or equal to 0.01 and in a lot of cases greater than or equal to 0.05. This difference in density is habitually less than or equal to 0.4, often less than or equal to 0.2 and frequently less than or equal to 0.1.

In the process according to the invention, the epichlorohydrin content in fraction (I) is generally greater than or equal to 600 g of epichlorohydrin per kg of fraction (I) and often greater than or equal to 700 g/kg. This content is usually less than or equal to 950 g of epichlorohydrin per kg of fraction (I) and often less than or equal to 800 g/kg.

In the process according to the invention, when the mixture from step (a) comprises at least one salt, the salt content in fraction (II) is generally greater than or equal to 5 g of salt per kg of fraction (II), usually greater than or equal to 30 g/kg, often greater than or equal to 50 g/kg, in a lot of cases greater than or equal to 100 g/kg and frequently greater than or equal to 150 g/kg. This salt content is usually less than or equal to 270 g of salt per kg of fraction (II), generally less than or equal to 250 g, in a lot of cases less than or equal to 240 g/kg, frequently less than or equal to 220 g/kg and often less than or equal to 200 g/kg.

In the process according to the invention, the water content in fraction (II) is generally greater than or equal to 700 g of water per kg of fraction (II), usually greater than or equal to 720 g/kg, frequently greater than or equal to 740 g/kg and often greater than or equal to 750 g/kg. This water content is usually less than or equal to 995 g of water per kg of fraction (II), usually less than or equal to 950 g/kg, frequently less than or equal to 900 g/kg and often less than or equal to 850 g/kg. In the process according to the invention, the volumes V_{I} and V_{II} of fractions (I) and (II) may be adjusted by any means. It is possible, for example, to independently adjust the total height of liquid in the phase separation zone and the height of the interface between fractions (I) and (II).

The total height of liquid may, for example, be adjusted by setting the overflow level of the phase separation zone with a dip tube or with a bottom valve coupled to a level detector. This level detector may be based on any type of level measurement method, such as hydrostatic methods with a float, plunger, electromagnetic sensor, pressure sensor or bubble sensor, electrical level measurement methods with conductive probes or capacitive probes and methods based on the use of radiation with ultrasonic probes, radar and optical probes.

The height of the interface may be adjusted for example using an adjustable gooseneck or via differential level measurements using the methods described above.

In the process according to the invention, a preferred way of adjusting the volumes V_{I} and V_{II} consists in adjusting the total height of liquid in the separation zone via an overflow and the height of the interface between fractions (I) and (II) via a bottom valve coupled to a level detector.

In the process according to the invention, the draw-off flow rates D_{I} and D_{II} of fractions (I) and (II) may be adjusted by any means for measuring liquid flow rate coupled to any draw-off means. The means for measuring flow rate are, for example, via thermal mass flow meters, Coriolis mass flow meters, ultrasonic flow meters, electromagnetic flow meters, float flow meters, differential pressure flow meters, volumetric flow meters, turbine flow meters and vortex flow meters. The draw-off means are, for example, via pumps, gravity feeds with a gooseneck or gravity feeds with a valve.

In the process according to the invention, a preferred way of adjusting the draw-off flow rates D_{I} and D_{II} is to use a gravity means for the light phase and a gravity means with a valve for the heavy phase.

The fraction (I) drawn off in the process according to the invention may be subjected to at least one subsequent treatment chosen from the group constituted of dilution, concentration, evaporation, distillation, stripping, liquid/liquid extraction and adsorption, and combinations of at least two thereof. This treatment may be as described in application WO 2008/152045 in the name of SOLVAY (Societe Anonyme), of which the content, and more specifically the passage from page 17, line 20, to page 23, line 5, is incorporated herein by reference.

The fraction (II) drawn off in the process according to the invention may be subjected to at least one subsequent treatment chosen from the group constituted of a physical treatment, a chemical treatment, a biological treatment, and combinations of at least two thereof. The physical treatment may be chosen from the group constituted of dilution, concentration, evaporation, distillation, stripping, liquid/liquid extraction, filtration and adsorption operations, alone or in combination. The chemical treatment may be chosen from the group constituted of oxidation, reduction, neutralization, complexation and precipitation operations, alone or in combination. The biological treatment may be chosen from the group constituted of aerobic or anaerobic bacterial treatments, alone or in combination. The bacteria may be free (activated sludge, lagooning) or fixed (bacteria bed, planted filters, sand filters, biofilter) or else biodiscs. These treatments may be as described in application WO 2008/152043 in the name of SOLVAY (Societe Anonyme), of which the content, and more specifically the passage from page 11, line 13, to page 29, line 7, is incorporated herein by reference.

Examples 1 to 12 below are intended to illustrate the invention without however limiting it.

### Example 1 (in accordance with the invention)

Introduced into a gravity settling tank are 1000 kg/h of a mixture of an aqueous phase and of an organic phase containing 225 g of epichlorohydrin/kg, 62 g of dichloropropanol/kg and 140 g of NaCl/kg. The mixture has a pH of 7. The settling tank functions at 40° C. and under autogenous pressure of the system. The settling tank is design to have an hold up of the aqueous phase of 0.054 m³ and an hold-up of the organic phase of 0.214 m³. The flows and the compositions of the phases leaving the settling tank are calculated using ASPEN+ and Aspen Tech software, taking into account the hydrolysis reactions that take place in each of the phases present. The epichlorohydrin loss by chemical reaction at the outlet of the settling tank is calculated and the results are given in table 1.

### Example 2, 3, 4 and 9 (not in accordance with the invention)

The procedure from Example 1 is followed, except that the settling is carried out so as to ensure a defined hold-up of the aqueous and the organic phase. The epichlorohydrin loss by chemical reaction at the outlet of the settling tank is calculated and the results are given in table 1.

### Example 5, 6, 7 and 8 (in accordance with the invention)

The procedure from Example 1 is followed, except that the settling is carried out so as to ensure a defined hold-up of the aqueous and the organic phase. The epichlorohydrin loss by chemical reaction at the outlet of the settling tank is calculated and the results are given in table 1.

### Example 10 and 11 (in accordance with the invention)

The procedure from Example 1 is followed, except that the settling is carried out at 30 ° C. so as to ensure a defined hold-up of the aqueous and the organic phase. The epichlorohydrin loss by chemical reaction at the outlet of the settling tank is calculated and the results are given in table 1.

### Example 12 (not in accordance with the invention)

The procedure from Example 1 is followed, except that the settling is carried out at 30 ° C. so as to ensure a defined hold-up of the aqueous and the organic phase. Theepichlorohydrin loss by chemical reaction at the outlet of the settling tank is calculated and the results are given in table 1.

| | | | | | epichlorohydrin | | |
|---|---|---|---|---|---|---|---|
| | VII | VI | DII | DI | loss | | Accordance |
| Example | m³ | m³ | m³/h | m³/h | g/h | % | invention |
| 1 | 0.054 | 0.214 | 0.646 | 0.233 | 33 | −0.015 | Yes |
| 2 | 0.323 | 0.116 | 0.646 | 0.233 | 136 | −0.060 | No |
| 3 | 0.592 | 0.019 | 0.646 | 0.233 | 244 | −0.108 | No |
| 4 | 0.485 | 0.155 | 0.646 | 0.233 | 204 | −0.091 | No |
| 5 | 0.108 | 0.116 | 0.646 | 0.233 | 51 | −0.023 | Yes |
| 6 | 0.162 | 0.116 | 0.646 | 0.233 | 70 | −0.031 | Yes |
| 7 | 0.1514 | 0.058 | 0.646 | 0.233 | 63 | −0.028 | Yes |
| 8 | 0.108 | 0.194 | 0.646 | 0.233 | 53 | −0.023 | Yes |
| 9 | 0.538 | 0.039 | 0.646 | 0.233 | 222 | −0.099 | No |
| 10 | 0.321 | 0.192 | 0.642 | 0.231 | 54 | −0.024 | Yes |
| 11 | 0.053 | 0.211 | 0.642 | 0.231 | 13 | −0.006 | Yes |
| 12 | 0.588 | 0.019 | 0.642 | 0.231 | 93 | −0.041 | No |

1. A process for manufacturing epichlorohydrin, comprising:
(a) preparing epichlorohydrin to obtain a mixture comprising epichlorohydrin and water;
(b) subjecting the mixture obtained in step (a) to a liquid-liquid phase separation to separate at least one first fraction (I) comprising most of the epichlorohydrin contained in the mixture obtained in step (a) before the separation and at least one second fraction (II) comprising most of the water contained in the mixture obtained in step (a) before the separation; and
(c) drawing off said first fraction (I) and said second fraction (II);
wherein the volume V_{I} of the first fraction (I) obtained in step (b) expressed in m³, the volume V_{II} of the second fraction (II) obtained in step (b) expressed in m³, the draw-off flow rate D_{I} of the first fraction (I) in step (c) expressed in m³/h, and the draw-off flow rate IN of the second fraction (II) in step (c) expressed in m³/h, correspond to the following formula:
(V_{II}/V_{I})<(D_{II}/D_{I})

2. The process according to claim 1, wherein
(V_{II}/V_{I})<0.7 (D_{II}/D_{I})

3. The process according to claim 2, wherein
(V_{II}/V_{I})<0.5 (D_{II}/D_{I})

4. The process according to claim 3, wherein
(V_{II}/V_{I})<0.1 (D_{II}/D_{I})

5. The process according to claim 1, wherein the volume V_{I} of the first fraction (I) obtained in step (b) expressed in m³, the volume V_{II} of the second fraction (II) obtained in step (b) expressed in m³, the draw-off flow rate D_{I} of the first fraction (I) in step (c) expressed in m³/h, and the draw-off flow rate D_{II} of the second fraction (II) in step (c) expressed in m³/h, correspond to the following formula:
[(V_{I}+V_{II})/(D_{I}+D_{II})]<10 h

6. The process according to claim 5, wherein
[(V_{I}+V_{II})/(D_{I}+D_{II})]<1 h

7. The process according to claim 6, wherein
[(V_{I}+V_{II})/(D_{I}+D_{II})]<0.5 h

8. The process according to claim 1, wherein said mixture obtained in step (a) further comprises at least one salt; wherein said at least one salt is sodium chloride; and wherein the sodium chloride content in said mixture obtained in step (a) is greater than or equal to 120 g of NaCl per kg of said mixture.

9. (canceled)

10. The process according to claim 8, wherein the sodium chloride content in said second fraction (II) is greater than or equal to 5 g of NaCl per kg of said second fraction (II).

11. The process according to claim 10, wherein the sodium chloride content in said second fraction (II) is greater than or equal to 50 g of NaCl per kg of said second fraction (II).

12. The process according to claim 11, wherein the sodium chloride content in said second fraction (II) is greater than or equal to 150 g of NaCl per kg of said second fraction (II).

13. The process according to claim 1, wherein the epichlorohydrin content in said mixture obtained in step (a) is greater than or equal to 200 g of epichlorohydrin per kg of said mixture.

14. The process according to claim 1, wherein the epichlorohydrin content in said first fraction (I) is greater than or equal to 600 g of epichlorohydrin per kg of said first fraction (I).

15. The process according to claim 1, wherein said mixture obtained in step (a) further comprises dichloropropanol.

16. The process according to claim 15, wherein the dichloropropanol content in the mixture obtained in step (a) is greater than or equal to 50 g of dichloropropanol per kg of said mixture.

17. The process according to claim 1, wherein said liquid-liquid phase separation of step (b) is carried out by unassisted gravitation.

18. The process according to claim 1, wherein the difference in density between the drawn off first fraction (I) and second fraction (II) is greater than or equal to 0.001 and less than or equal to 0.4.

19. The process according to claim 1, wherein said mixture obtained in step (a) originates partly from a process for manufacturing epichlorohydrin by alkaline dehydrochlorination of dichloropropanol, at least one portion of said dichloropropanol being obtained from glycerol, and at least one fraction of said glycerol being natural glycerol.

20. The process according to claim 19, wherein dichloropropanol is added to said mixture obtained in step (a).

21. The process according to claim 1, wherein the pH of said mixture obtained in step (a) is controlled and maintained at a value greater than or equal to 4 and less than or equal to 10.
